(19) United States
(12) Patent Application Publication
(10) Pub. No.: US 2024/0360395 A1
(43) Pub. Date: Oct. 31, 2024

(54) POLYSTYRENE COATED CELL CULTURE SUBSTRATES, FIXED BED BIOREACTORS, AND RELATED METHODS

(71) Applicant: CORNING INCORPORATED, CORNING, NY (US)

(72) Inventor: Ye Fang, Painted Post, NY (US)

(21) Appl. No.: 18/692,663

(22) PCT Filed: Sep. 9, 2022
(86) PCT No.: PCT/US2022/042985

§ 371 Date: Mar. 15, 2024

Related U.S. Application Data
(60) Provisional application No. 63/244,482, filed on Sep. 15, 2021.

(51) Int. Cl. C12M 1/00; C12M 1/12

(52) U.S. Cl. CPC C12M 23/20; C12M 25/18

(57) ABSTRACT

A bioreactor system for culturing cells is provided. The system includes a cell culture vessel with at least one interior reservoir for flowing liquid media therethrough. The system also includes a cell culture matrix disposed in the reservoir, the cell culture matrix having a substrate with a polymer base and a polystyrene coating disposed on the polymer base.

### CROSS-REFERENCE TO RELATED APPLICATIONS

This application claims the benefit of priority under 35 U.S.C. § 119 of U.S. Provisional Application Ser. No. 63/244,482 filed on Sep. 15, 2021, the content of which is relied upon and incorporated herein by reference in its entirety.

### FIELD OF THE DISCLOSURE

This disclosure general relates to substrates for culturing cells in fixed bed bioreactors. In particular, the present disclosure relates to polystyrene substrates for fixed bed reactor, including coatings, and related methods.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Adherent cell culture is dominating the production of viral vectors for gene and modified cell therapy. This is because cells used for viral vector production are mostly anchorage-dependent. Viral vectors are commonly used to deliver genetic materials into cells and tissues so that genetic defects can be corrected, cellular and tissue function be enhanced, or the production of cellular products be improved, ultimately leading to potential curative treatment. Adherent cell culture is also dominating scale up of stem cells for regenerative medicine. This is because stem cells such as induced pluripotent stem cells (iPSCs) and mesenchymal stem cells (MSCs) are also inherently anchorage-dependent. Stem cells hold great promise for cell therapy, tissue engineering, and regenerative medicine as well as pharmaceutical and biotechnological applications.

There is a strong need for reliable and efficient platforms to scale up adherent cell culture. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing.

Another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Many existing fixed bed bioreactors use polyethylene terephthalate (PET) as the substrate material (e.g., PET microfibers; PET non-woven webs), which are primarily used for viral vector and vaccine production but their other applications for adherent cell culture are limited. For example, many of the existing fixed bed bioreactors have not been used for stem cell culture. Therefore, there is a strong need to develop a fixed bed made of materials that can support the culture of a broader range of cell types.

Mammalian cells are used to produce therapeutic proteins, monoclonal antibodies, viral vectors, and even cultured meat. Furthermore, in tissue engineering and regenerative medicine billions of stem cells are used to fabricate tissue engineered constructs or to replenish lost or damaged cells in degenerative diseases. Although suspension cell culture is widely used to produce proteins and antibodies, adherent cell culture is dominating the production of viral vectors for gene and modified cell therapy, as well as stem cells for regenerative medicine. Viral vectors are commonly used to deliver genetic materials into cells and tissues so that genetic defects can be corrected, cellular and tissue function be enhanced, or the production of cellular products be improved, ultimately leading to potential curative treatment.

Stem cells hold great promise for cell therapy, tissue engineering, and regenerative medicine as well as pharmaceutical and biotechnological applications. However, cells used for viral vector production are mostly anchorage-dependent; similarly, stem cells such as induced pluripotent stem cells (iPSCs) and mesenchymal stem cells (MSCs) are also inherently anchorage-dependent. Therefore, there is a strong need for a reliable and efficient platform to scale up adherent cell culture.

Polystyrene has been used as a cell culture surface for many years (Lerman, M. J et al. “The evolution of polystyrene as a cell culture material” Tissue Engineering B 2018, 24, 359-372). Polystyrene (PS) is a clear, amorphous, nonpolar thermoplastic that is easy to process and that can be easily fabricated into a number of finished goods since it is a viscous liquid above its glass transition temperature that can be easily molded. Furthermore, although native PS surfaces poorly facilitate cell adhesion and growth in vitro, a wide variety of liquid surface deposition, energetic plasma activation, and other functionalization methods have been developed to transform the surface chemistry, driving PS to be the de facto substrate for cell culture. However, it is difficult to use polystyrene to make woven or non-woven meshes or fabrics, because polystyrene is a brittle plastic material and has poor impact strength due to the stiffness of the polymer backbone. Therefore, there is a need to develop PS coated plastic meshes or fabrics to produce fixed beds used in bioreactors for adherent cell culture.

There is a need for cell culture substrates and/or matrices, bioreactors, systems, and methods that enable culturing of cells in a high-density format, with uniform cell distribution, and easily attainable and increased harvesting yields, and with the flexibility to be used in multiple cell culture applications by using polystyrene-based materials adapted into a high-density format such as woven or non-woven meshes or fabrics.

### SUMMARY

According to embodiments of this disclosure, a bioreactor system for culturing cells is provided. The bioreactor system includes a cell culture vessel including at least one interior reservoir configured for flowing liquid media therethrough; and a cell culture matrix disposed in the reservoir. The cell culture matrix includes a substrate having a polymer base and a polystyrene coating disposed on the polymer base. As an aspect of embodiments, the polystyrene coating is functionalized to enhance cell attachment and/or growth thereto. The polystyrene coating can be plasma treated. The substrate can further include a cell attachment treatment on the polystyrene coating. The cell attachment treatment can include at least one of an oxygen plasma, an allylamine coating, a carboxylic acid group, or a biologics coating. The biologics coating can include at least one of laminin, fibronectin, extracellular proteins, and synthetic peptides. The polymer base can include a thermopolymer. Examples of the polymer base include at least one of polyethylene terephthalate, polypropylene, nylon, polylactic acid, polybenzimidazole, polycarbonate, polyether sulfone, polyoxymethylene, polyetherether ketone, polyethylene, polyphenylene oxide, polyphenylene sulfide, polyvinyl chloride, and polyvinylidene fluoride.

An aspect of embodiments includes the substrate having woven or non-woven fibers. The cell culture matrix can include a plurality of layers of substrate. In embodiments, the plurality of layers is in a stacked arrangement. In embodiments, the substrate is a rolled substrate. The substrate can include fibers with a fiber diameter of from about 10 micrometers to about 500 micrometers. The substrate has a regular array of openings between fibers of the substrate. The substrate comprises an effective porosity of about 10% to about 80%. The substrate is configured for at least one of viral vector production, induced pluripotent stem cells (iPSCs), mesenchymal stem cells (MSCs), protein production, antibody production, and extracellular vesicle production.

### BRIEF DESCRIPTION OF THE DRAWINGS

FIG. 1A shows a perspective view of a three-dimensional model of a cell culture substrate, according to embodiments of this disclosure.

FIG. 1B is a two-dimensional plan view of the substrate of FIG. 1A.

FIG. 1C is a cross-section along line A-A of the substrate in FIG. 1B.

FIG. 2 shows a schematic view of a cell culture system, according to embodiments.

FIG. 3 shows a schematic view of a cell culture system, according to embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

The present disclose relates to embodiments of a high-density adherent cell culture substrate and bioreactor vessels containing a fixed bed made of such substrate, with substrate being woven or non-woven plastic mesh/fabric substrates coated with polystyrene. The polystyrene coating is achieved using plasma polymerization process. The fixed bed can be made of woven or non-woven plastic mesh/fabric substrate materials. The polystyrene coating can be further treated using oxygen plasma (CellBind®), allylamine (PureAmine), or biologics including laminins, fibronectin or Corning Synthemax synthetic peptides.

Preexisting fixed bed bioreactors often use PET as the substrate (e.g., PET microfibers or PET non-woven webs), thus limiting their applications for adherent cell culture. Embodiments of the present disclosure include a surface of a polystyrene layer coated on any woven or non-woven plastic substrate, mesh, or fabric, and can leverage all well-established surface modifications of polystyrene substrates including CellBind®, amine or carboxylate coating, thus broadening the applications of fixed bed bioreactors to many different types of cells including, for example, stem cells.

Embodiments of this disclosure include cell culture substrates and cell culture bioreactors using cell culture substrates that include polystyrene (PS) substrate materials. Embodiments include polystyrene and composite or coated polystyrene substrate materials, as well as woven and non-woven structured substrates, sometimes referred to as meshes or fabrics, for high density adherent cell culture. The polystyrene coating is achieved using plasma polymerization process. The polystyrene coating can be further treated using oxygen plasma (CellBind®), allylamine (PureAmine), or biologics including laminins, fibronectin or Corning Incorporated's Synthemax® synthetic peptides. The cell culture vessels can be used for high density adherent culture of a wide variety of types of cells including stem cells.

Embodiments also include methods to coat or otherwise modify the surface of such substrates with a variety of surface chemistries to enhance cell culture. Such surface modifications include amine presenting surfaces to extracellular matrix protein coated surfaces, depending on the cell types and the use of the bioreactor. Embodiments also include bioreactor systems with different packed bed configurations and methods of packing substrates into different configurations of fixed beds, which can depend on the volumetric cell density to be achieved and the medium flow pattern. Embodiments of these cell culture vessels, substrates, and methods can be used for high density adherent culture of a wide variety of types of cells including stem cells, targeting applications ranging from viral vector production to proteins/antibody production, stem cell culture and reprogramming, and extracellular vesicle production.

Embodiments of this disclosure includes substrates, bioreactors, and methods having improved flow characteristics through the substrate and/or bioreactor vessel. For example, more uniform flow is achieved through the substrate, and non-uniform flow resulting from channeling or turbulent flow is reduced or eliminated. Flow “dead zones” in the substrate or fixed bed of the bioreactor are greatly reduced or eliminated compared to existing solutions. The result is a substrate or fixed bed that allows for uniform perfusion throughout the substrate or fixed bed, which promotes cell health during cell culture and an efficient cell culture process in terms of not only the culturing of cells, but also cell seeding, and harvesting of cells or cell by-products.

Cell harvesting is yet another problem encountered when bioreactors packed with non-woven fibrous scaffolds or disordered substrates are used. Due to the packed-bed functioning as a depth filter, cells that are released at the end of cell culture process are trapped inside the packed bed, and cell recovery is very low. This significantly limits utilization of such bioreactors in bioprocesses where live cells are the products or where live cells need to be harvested for further processing to capture cell by-products. Thus, the non-uniformity leads to areas with different exposure to flow and shear, effectively reducing the usable cell culture area, causing non-uniform culture, and interfering with transfection efficiency and cell release. Thus, embodiments of this disclosure include substrates, bioreactors, and methods that enable high-yield harvesting of viable cells.

To address these and other problems of existing cell culture solutions, embodiments of the present disclosure provide cell growth substrates, matrices including such substrates, and/or packed-bed systems using such substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution.

In embodiments, a matrix is provided with a structurally defined surface area for adherent cells to attach and proliferate that has good mechanical strength and forms a highly uniform multiplicity of interconnected fluidic networks when assembled in a packed bed or other bioreactor. In embodiments, a mechanically stable, non-degradable ordered mesh can be used as the substrate to support adherent cell production. The mesh can be woven or non-woven. In the case of a wove substrate, fibers or bundles of fibers are interwoven according one or more weave patterns known in the art. A non-woven substrate can be achieved in a number of ways. While non-woven substrates exist in the marketplace, those solutions use randomly oriented fibers or spun fibers. Preferred embodiments of the present disclosure use structurally defined substrates with planned and ordered structural arrays of fibers. Thus, in the case of non-woven substrates, the fibers still have a defined and ordered structure. Nonwoven substrates can be formed by 3D printing of the substrate material or fibers into an organized shape; by bonding or adhering fibers into a mesh or similar fabric-like material; or by other methods of combining fibers known to those of ordinary skill in the art. While some aspects discussed herein refer to woven or nonwoven aspects of embodiments, it should be appreciated that, unless otherwise stated, either method (woven or non-woven) can be used to create similarly ordered, structurally defined substrates. Thus, the use of “woven” or “non-woven” here is not intended to limit those aspects to a woven or non-woven aspect of embodiments of this disclosure.

The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of embodiments enables complete cell recovery and consistent cell harvesting from the packed bed of the bioreactor.

According to embodiments, a method of cell culturing is also provided using bioreactors with the matrix for bioprocessing production of therapeutic proteins, antibodies, viral vaccines, stem cells, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In embodiments, the substrate is a polymer-based material with a thermopolymer base layer and a polystyrene coating material, and can be formed as a molded polymer sheet; a polymer sheet with openings punched through the thickness; a number of filaments that are fused into a mesh-like layer; a 3D-printed substrate; or a plurality of filaments that are woven into a mesh layer, for example. However, embodiments are not limited to substrates formed in these ways, which are provided as examples of aspects of embodiments. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

In addition, embodiments of this disclosure enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

Embodiments of this disclosure include substrates made of any thermopolymer. The only limitation on the type of thermopolymer used is that it possible to make the form of structurally defined meshes, clothes, webs, fabrics, or other ordered substrates according to embodiments of this disclosure. The thermopolymer itself does not need to be compatible or supportive to cell attachment or culture. Examples of thermopolymers include polyethylene terephthalate (PET), polypropylene, nylon, polylactic acid, polybenzimidazole, polycarbonate, polyether sulfone, polyoxymethylene, polyetherether ketone, polyethylene, polyphenylene oxide, polyphenylene sulfide, polyvinyl chloride, polyvinylidene fluoride, and the like. All these polymers can be used to make mesh or web structures.

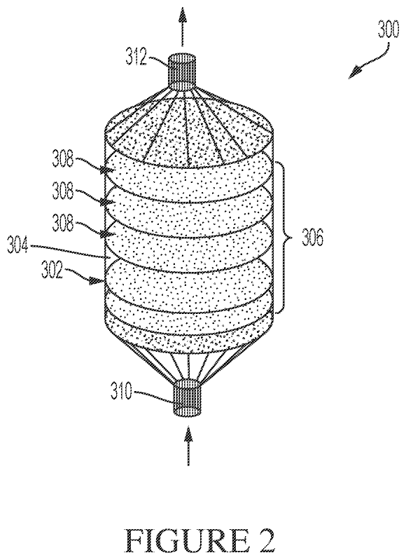

According to embodiments, substrates, such as woven or non-woven meshes, are coated with a layer of polystyrene. In embodiments using a stack of substrate layers (see FIG. 2), the individual layers of substrate can be individually coated. The thickness of the polystyrene layer can be, for example, 5 nm, 25 nm, 50 nm, 200 nm, 500 nm, 1 um, or 5 um. The polystyrene layer coating is achieved using a dry process, such as plasma polymerization, as described in literature (Luo, H. L. et al. Plasma polymerization of styrene with carbon dioxide under glow discharge conditions Appl. Surf. Sci. 2007, 253, 5203-5207. Tan, P. E. C. et al. Plasma polymerization of styrene using an argon-fed atmospheric pressure plasma jet J. Vac. Sci. Technol. B 2018, 36, 041102; Choudhury, A. J., et al. Synthesis and characterization of plasma polymerized styrene films by rf discharge. J. Phys. Conf. Ser. 2010, 208, 012104).

The surface of the polystyrene layer on the plastic meshes can be further modified to support cell attachment and growth. The choice of surface modification is dependent on cell type and applications. For instance, oxygen plasma treatment can be used to convert the polystyrene surface into a cell bind surface. Plasma treatment of the polystyrene layer with allylamine can convert the surface to present amine functional groups, while plasma treatment with acrylic acid can convert the surface to present carboxylic acid groups, both of which can support the attachment and growth of certain types of cells. These surface modification processes are well established, as described in literature (Kurosawa, S., et al. Behavior of contact angle on glass plates coated with plasma polymerized styrene, allylamine and acrylic acid. J. Photopolymer Sci. Technol. 1999, 12, 63-68; Choukourov, A., et a. Mechanistic studies of plasma polymerization of allylamine. J. Phys. Chem. 2005, 109, 23086-23095). In addition, the amine or carboxylic acid groups can be further used to conjugate cell attachment enhancement biologics such as collagen, gelatin, laminins, fibronectin or Corning Synthemax vitronectin peptides using state of the art bioconjugation chemistry methods. These biologics modified surfaces are particularly useful for stem cell culture.

Embodiments of the present disclosure include composite polystyrene substrates, including woven and non-woven substrates, to make fixed beds for bioreactors used as the vessels for high-density adherent cell culture. Embodiments also include methods to coat such substrates, providing a wide range of available surface chemistries to enhance cell culture. These include amine presenting polymers, extracellular proteins such as collagen, elastin, fibronectin, laminin, synthetic peptides such as Synthemax® vitronectin peptides, and hydrogels. Embodiments also include methods for packing or assembling such substrates into different configurations for the fixed beds, which can depend on the volumetric cell density to be achieved and the medium flow pattern to be sought.

In embodiments, the polystyrene composite substrates are woven. The type of weave can vary, and any known weave pattern can be used in embodiments to make fixed beds within a bioreactor for adherent cell culture. The woven polystyrene or polystyrene composite substrates can be made of fibers where each fiber can have a diameter of about 10 micrometers to 500 micrometers, or up to 1,000 micrometers or greater. The woven substrates can have a regular gap between adjacent fibers to create openings which allow flow of cell culture media, cells, nutrients, and cell by-products to pass through the openings. The woven fabric substrate has an effective porosity in range of 10% to 80%. When the glass fiber has a small diameter (e.g., 10 micrometers to 30 micrometers; or 10 micrometers to 100 micrometers), several glass fibers can be bundled tightly together to form a joint fiber or a bundled fiber. The surface of this bundled fiber is suitable for cell adhesion and growth.

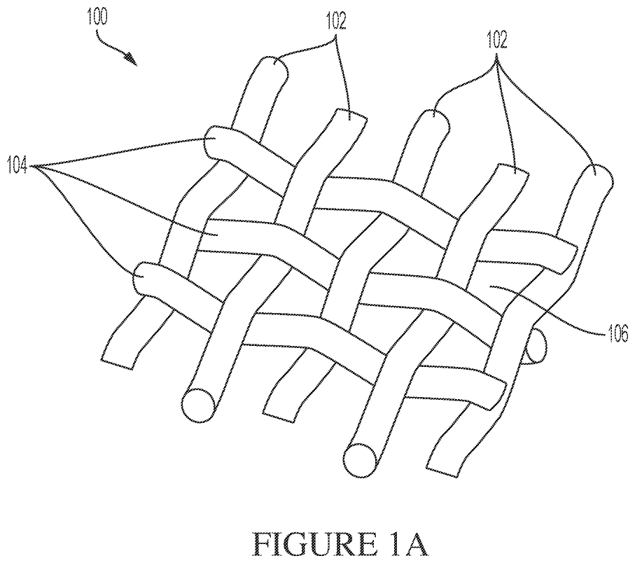

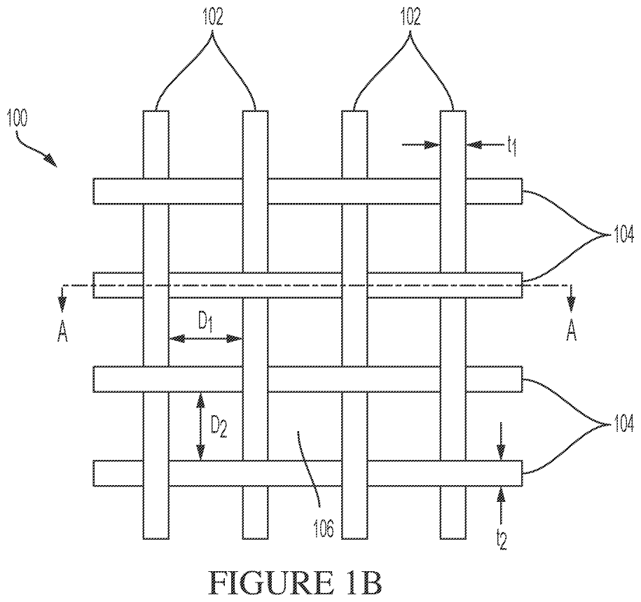

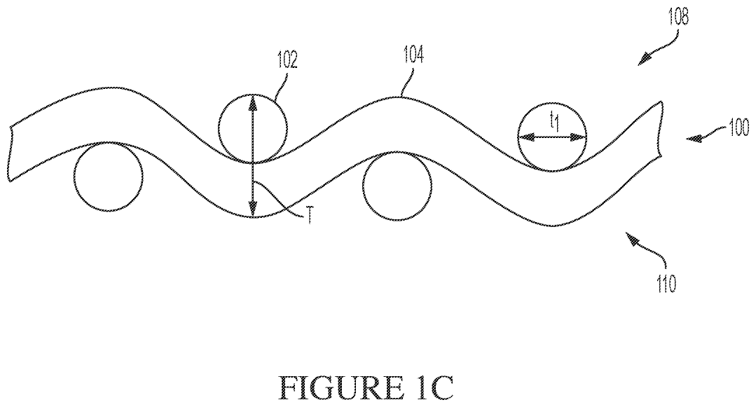

FIGS. 1A and 1B show a three-dimensional (3D) perspective view and a two-dimensional (2D) plan view, respectively, of a cell culture substrate 100, according to an example of embodiments of this disclosure. The cell culture substrate 100 is a woven mesh layer made of a first plurality of fibers 102 running in a first direction and a second plurality of fibers 104 running in a second direction. The woven fibers of the substrate 100 form a plurality of openings 106, which can be defined by one or more widths or diameters (e.g., D₁, D₂). The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Thus, as shown in FIG. 1C, a thickness T of the woven mesh 100 may be thicker than the thickness of a single fiber (e.g., t₁). As used herein, the thickness T is the maximum thickness between a first side 108 and a second side 110 of the woven mesh. Without wishing to be bound by theory, it is believed that the three-dimensional structure of the substrate 100 is advantageous as it provides a large surface area for culturing adherent cells, and the structural rigidity of the mesh can provide a consistent and predictable cell culture matrix structure that enables uniform fluid flow. While the example of FIGS. 1A and 1B shows the woven aspect of embodiments of this disclosure, the above and following descriptions of the structure and geometry also applies to non-woven aspects of embodiments.

In FIG. 1B, the openings 106 have a diameter D₁, defined as a distance between opposite fibers 102, and a diameter D₂, defined as a distance between opposite fibers 104. D₁ and D₂ can be equal or unequal, depending on the weave geometry. Where D₁ and D₂ are unequal, the larger can be referred to as the major diameter, and the smaller as the minor diameter. In embodiments, the diameter of an opening may refer to the widest part of the opening. Unless otherwise specified, the opening diameter, as used herein, will refer to a distance between parallel fibers on opposite sides of an opening.

A given fiber of the plurality of fibers 102 has a thickness t₁, and a given fiber of the plurality of fibers 104 has a thickness t₂. In the case of fibers of round cross-section, as shown in FIG. 1A, or other three-dimensional cross-sections, the thicknesses t₁ and t₂ are the maximum diameters or thicknesses of the fiber cross-section. According to embodiments, the plurality of fibers 102 all have the same thickness t₁, and the plurality of fiber 104 all have the same thickness t₂. In addition, t₁ and t₂ may be equal. However, in embodiments, t₁ and t₂ are not equal such as when the plurality of fibers 102 are different from the plurality of fiber 104. In addition, each of the plurality of fibers 102 and plurality of fibers 104 may contain fibers of two or more different thicknesses (e.g., t₁ₐ, t_{1b}, etc., and t₂ₐ, t_{2b}, etc.). According to embodiments, the thicknesses t₁ and the are large relative to the size of the cells cultured thereon, so that the fibers provide an approximation of a flat surface from the perspective of the cell, which can enable better cell attachment and growth as compared to some other solutions in which the fiber size is small (e.g., on the scale of the cell diameter). Due to three-dimensional nature of woven mesh, as shown in FIGS. 1A-1C, the 2D surface area of the fibers available for cell attachment and proliferation exceeds the surface area for attachment on an equivalent planar 2D surface. Although FIGS. 1A-1C show an example in which the substrate is a woven mesh, this woven aspect is provided only as an illustration of an example of embodiments of this disclosure. Embodiments are not limited to this woven aspect, and the substrate can be formed via other means described herein, including by 3D printing, or combining fibers under one or more of heat and pressure to fuse fibers, or fibers adhered to each other through other means.

In embodiments, a fiber may have a diameter in a range of about 10 μm to about 1000 μm; about 10 μm to about 50 μm; about 10 μm to about 30 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be made into a mesh with openings ranging from about 100 μm×100 μm to about 1000 μm×1000 μm. In embodiments, the opening may have a diameter to about 50 μm to about 1000 μm; about 100 μm to about 750 μm; about 125 μm to about 600 μm; about 150 μm to about 500 μm, about 200 μm to about 400 μm; or about 200 μm to about 300 μm. These ranges of the filament diameters and opening diameters are examples of embodiments, but are not intended to limit the possible feature sizes of the mesh according to embodiments. The combination of fiber diameter and opening diameter is chosen to provide efficient and uniform fluid flow through the substrate when, for example, the cell culture matrix includes a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g, when maximizing substrate surface area is a priority). According to embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, is single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area. According to embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and embodiments may have different effective surface areas. The cell culture matrix can also be characterized in terms of porosity, as discussed in the Examples herein.

The substrate can be fabricated from monofilament or multifilament fibers of glass or polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide, or fiber glass and fiberglass composite materials, including polymer-coated fiberglass. Mesh substrates may have a different patterns or weaves, including, for example knitted, warp-knitted, or woven (e.g., plain weave, twilled weave, dutch weave, five needle weave).

By using a structurally defined culture matrix of sufficient rigidity, high-flow-resistance uniformity across the matrix or packed bed is achieved. According to embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

The geometry of the mesh substrate layers is designed to allow efficient and uniform flow through one or multiple substrate layers. In addition, the structure of the matrix can accommodate fluid flow through the matrix in multiple orientations. For example, the direction of bulk fluid flow can be perpendicular to the major side surfaces of the first and second substrate layers. However, the matrix can also be oriented with respect to the flow such that the sides of the substrate layers are parallel to the bulk flow direction. In addition to fluid flow being perpendicular or parallel to the first and second sides of the mesh layers, the matrix can be arranged with multiple pieces of substrate at intermediate angles, or even in random arrangements with respect to fluid flow. This flexibility in orientation is enabled by the essentially isotropic flow behavior of the woven substrate. In contrast, substrates for adherent cells in existing bioreactors do not exhibit this behavior and instead their packed beds tend to create preferential flow channels and have substrate materials with anisotropic permeability. The flexibility of the matrix of the current disclosure allows for its use in various applications and bioreactor or container designs while enabling better and more uniform permeability throughout the bioreactor vessel.

As discussed herein, the cell culture substrate can be used within a bioreactor vessel, according to embodiments. For example, the substrate can be used in a packed bed bioreactor configuration, or in other configurations within a three-dimensional culture chamber. However, embodiments are not limited to a three-dimensional culture space, and it is contemplated that the substrate can be used in what may be considered a two-dimensional culture surface configuration, where the one or more layers of the substrate lay flat, such as within a flat-bottomed culture dish, to provide a culture substrate for cells. Due to contamination concerns, the vessel can be a single-use vessel that can be disposed of after use.

A cell culture system is provided, according to embodiments, in which the cell culture matrix is used within a culture chamber of a bioreactor vessel. FIG. 2 shows an example of a cell culture system 300 that includes a bioreactor vessel 302 having a cell culture chamber 304 in the interior of the bioreactor vessel 302. Within the cell culture chamber 304 is a cell culture matrix 306 that is made from a stack of substrate layers 308. The substrate layers 308 are stacked with the first or second side of a substrate layer facing a first or second side of an adjacent substrate layer. The bioreactor vessel 300 has an inlet 310 at one end for the input of media, cells, and/or nutrients into the culture chamber 304, and an outlet 312 at the opposite end for removing media, cells, or cell products from the culture chamber 304. By allowing stacking of substrate layers in this way, the system can be easily scaled up without negative impacts on cell attachment and proliferation, due to the defined structure and efficient fluid flow through the stacked substrates. While the vessel 300 may generally be described as having an inlet 310 and an outlet 312, embodiments may use one or both of the inlet 310 and outlet 312 for flowing media, cells, or other contents both into and out of the culture chamber 304. For example, inlet 310 may be used for flowing media or cells into the culture chamber 304 during cell seeding, perfusion, or culturing phases, but may also be used for removing one or more of media, cells, or cell products through the inlet 310 in a harvesting phase. Thus, the terms “inlet” and “outlet” are not intended to restrict the function of those openings.

In embodiments, flow resistance and volumetric density of the packed bed can be controlled by interleaving substrate layers of different geometries. In particular, mesh size and geometry (e.g., fiber diameter, opening diameter, and/or opening geometry) define the fluid flow resistance in packed bed format. By interlaying meshes of different sizes and geometries, flow resistance can be controlled or varied in one or more specific portions of the bioreactor. This will enable better uniformity of liquid perfusion in the packed bed. Various combinations of meshes of different sizes are possible to obtain different profiles of volumetric density of cells growth surface and flow resistance. For example, a packed bed column with zones of varying volumetric cells densities (e.g, a series of zones creating a pattern of low/high/low/high, etc. densities) can be assembled by interleaving meshes of different sizes.

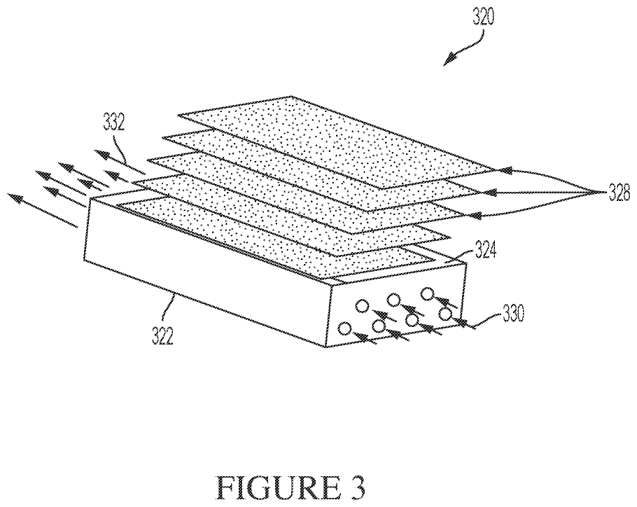

In FIG. 2, the bulk flow direction is in a direction from the inlet 310 to the outlet 312, and, in this example, the first and second major sides of the substrate layers 308 are perpendicular to the bulk flow direction. In contrast, the example shown in FIG. 3 is of an embodiment in which the system 320 includes a bioreactor vessel 322 and stack of substrates 328 within the culture space 324 that have first and second sides that are parallel to a bulk flow direction, which corresponds to a direction shown by the flow lines into the inlets 330 and out of the outlets 332. Thus, the matrices of embodiments of this disclosure can be employed in either configuration. In each of systems 300 and 320, the substrates 308, 328 are sized and shaped to fill the interior space defined by the culture chamber 304, 324 so that the culture spaces in each vessel are filled for cell growth surfaces to maximize efficiency in terms of cells per unit volume. Although FIG. 3 shows multiple inlets 330 and multiple outlets 332, it is contemplated that the system 320 may be fed by a single inlet and have a single outlet. However, according to embodiments herein, distribution plates can be used to help distribute the media, cells, or nutrients across a cross-section of the packed bed and thus improve uniformity of fluid flow through the packed bed. As such, the multiple inlets 330 represent how a distribution plate can be provided with a plurality of holes across the packed-bed cross-section for creating more uniform flow.

The cell culture matrix can be arranged in multiple configurations within the culture chamber depending on the desired system. For example, in embodiments, the system includes one or more layers of the substrate with a width extending across the width of a defined cell culture space in the culture chamber (as in FIG. 2). Multiple layers of the substrate may be stacked, as shown in FIG. 2, in this way to a predetermined height. As discussed above, the substrate layers may be arranged such that the first and second sides of one or more layers are perpendicular to a bulk flow direction of culture media through the defined culture space within the culture chamber, or the first and second sides of one or more layers may be parallel to the bulk flow direction. In embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In embodiments, the fixed bed can include one or more substrate layers that are wound into a roll, such as a cylindrical roll. The rolled bed can be made of a single layer of substrate that is rolled, or can include multiple layers of substrate. When using multiple layers of substrate, the multiple layers can be made from the same or different substrate materials. For example, a layer of open substrate (e.g., with openings through the layer for fluid flow) and closed substrate (e.g., with substantial no openings) can be laid on top of each other, and rolled together, forming a roll of alternating substrates. The other combinations of substrate stacks are possible in the roll. For example, two layers of an open substrate can be stacked on a single layer of closed substrate and then rolled.

In embodiments, the fixed bed can be made of a three-dimensional woven or non-woven cell culture matrix with a substrate material comprising PS-based material as discussed herein. For example, individual layers such as those shown in FIGS. 1A and 1B can be bonded or fused together, or a monolithic substrate can be 3D printed or formed by other means capable of fusing or attached fibers or polystyrene composite material.

One aspect of embodiments provides a bioreactor vessel in a roller bottle configuration. The culture chamber is capable of containing a cell culture matrix and substrate according to the embodiments described in this disclosure. In the roller bottle configuration, the bioreactor vessel may be operably attached to a means for moving the bioreactor vessel about a central longitudinal axis of the vessel. For example, the bioreactor vessel may be rotated about the central longitudinal axis. The rotation may be continuous (e.g., continuing in one direction) or discontinuous (e.g., an intermittent rotation in a single direction or alternating directions, or oscillating in back and forth rotational directions). In operation, the rotation of the bioreactor vessel causes movement of cells and/or fluid within the chamber. This movement can be considered relative with respect to the walls of the chamber. For example, as the bioreactor vessel rotates about its central longitudinal axis, gravity may cause the fluid, culture media, and/or unadhered cells to remain toward a lower portion of the chamber. However, in embodiments, the cell culture matrix is essentially fixed with respect to the vessel, and thus rotates with the vessel. In embodiments, the cell culture matrix can be unattached and free to move to a desired degree relative to the vessel as the vessel rotates. The cells may adhere to the cell culture matrix, while the movement of the vessel allows the cells to receive exposure to both the cell culture media or liquid, and to oxygen or other gases within the culture chamber.

By using a cell culture matrix according to embodiments of this disclosure, such as a matrix including a woven or mesh substrate, the roller bottle vessel is provided with an increased surface area available for adherent cells to attach, proliferate, and function. In particular, using a substrate of a woven mesh of monofilament polymer material within the roller bottle, the surface area may increase by of about 2.4 to about 4.8 times, or to about 10 times that of a standard roller bottle. As discussed herein, each monofilament strand of the mesh substrate is capable of presenting itself as 2D surface for adherent cells to attach. In addition, multiple layers of mesh can we arranged in roller bottle, resulting in increases of total available surface area ranging from about 2 to 20 times that of a standard roller bottle. Thus, existing roller bottle facilities and processing, including cell seeding, media exchange, and cell harvesting, can be modified by the addition of the improved cell culture matrix disclosed herein, with minimal impact on existing operation infrastructure and processing steps.

The bioreactor vessel optionally includes one or more outlets capable of being attached to inlet and/or outlet means. Through the one or more outlets, liquid, media, or cells can be supplied to or removed from the chamber A single port in the vessel may act as both the inlet and outlet, or multiple ports may be provided for dedicated inlets and outlets.

The packed bed cell culture matrix of embodiments can consist of the woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

The surface chemistry of the mesh filaments may need to be modified to provide desired cell adhesion properties. Such modifications can be made by the chemical treatment of the polymer material of the mesh, by grafting cell adhesion molecules to the filament surface, or by coating with a material to enhance cell adhesion and growth. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. Embodiments include polystyrene composite fabric substrates with surface chemistry including amine presenting polymers, extracellular proteins such as collagen, elastin, fibronectin, laminin, synthetic peptides such as Synthemax® vitronectin peptides, and hydrogels. In embodiments, however, the mesh is capable of providing an efficient cell growth surface without surface treatment.

In embodiments, the coating material for polystyrene composite substrates can be introduced during the glass fiber sizing step. For instance, a silane, for instance γ-aminopropyltriethoxysilane (APTES), γ-glycidoxypropyltrimethoxysilane (GPTMS), γ-methacryloxypropyltrimethoxysilane (MPTMS), or vinyltriethoxysilane (VTES), can be used as a sizing agent to coat glass fibers during glass fiber manufacturing process. The coating can be preserved during the substrate manufacturing step and follow-up processing steps. Glass fiber manufacturing typically involves a combination of extrusion and attenuation of molten glass. The glass melt flows under gravity from a melting furnace via the forehearth to an array of bushings made of, for example, a platinum/rhodium alloy. These bushings contain a geometric array of tipped orifices from 200 to as many as 8000. Bushing plates are heated electrically, and their temperature is precisely controlled to maintain a constant glass viscosity. The molten glass drops from the bushing tips and is rapidly attenuated into fine fibers. A fine mist of water may be sprayed onto the filaments just below the bushing. This water spray is often mistaken as the primary cooling of the fiber forming process, and although the sprays do contribute to the fiber cooling, most of the heat is lost from the fibers by convection. Size is applied to freshly pulled glass fibers using a sizing applicator normally positioned approximately 1 to 2 meters below the bushing plate.

In embodiments, the coating material can be introduced before or after the fixed bed is made. This can be done via an optional heat treatment to clean the fiber glass substrate, then coating by vapor deposition of a silane and/or a solution reaction.

In embodiments, the substrate is coated with a layer of amine presenting polymers. The amine presenting polymer layer is formed during glass fiber sizing step, as mentioned above. As another aspect of embodiments, the substrate is coated with extracellular proteins or peptides via covalently coupling. The covalent coupling can be achieved via different schemes. For instance, a layer of γ-glycidoxypropyltrimethoxysilane (GPTMS) is formed during glass fiber sizing step and used as a coupling agent. After the fixed bed is assembled, a solution containing the extracellular matrix protein or peptides is introduced to react with the GPTMS layer.

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

Embodiments are not limited to the vessel rotation about a central longitudinal axis. For example, the vessel may rotate about an axis that is not centrally located with respect to the vessel. In addition, the axis of rotation may be a horizontal or vertical axis.

Embodiments of this disclosure includes aspects of embodiments disclosed in U.S. patent application Ser. Nos. 16/781,685; and 16/781,723; and U.S. Provisional Patent Application No. 63/227,693, the contents of which are hereby incorporated herein in their entireties.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a bioreactor system for culturing cells, the system comprising: a cell culture vessel comprising at least one interior reservoir configured for flowing liquid media therethrough; and a cell culture matrix disposed in the reservoir, the cell culture matrix comprising a substrate comprises a polymer base and a polystyrene coating disposed on the polymer base.

Aspect 2 pertains to the bioreactor system of Aspect 1, wherein the polystyrene coating is functionalized to enhance cell attachment and/or growth thereto.

Aspect 3 pertains to the bioreactor system of Aspect 1 or Aspect 2, wherein the polystyrene coating is plasma treated.

Aspect 4 pertains to the bioreactor system of any one of Aspects 1-3, further comprising a cell attachment treatment on the polystyrene coating.

Aspect 5 pertains to the bioreactor system of Aspect 4, wherein the cell attachment treatment comprises at least one of an oxygen plasma, an allylamine coating, a carboxylic acid group, or a biologics coating.

Aspect 6 pertains to the bioreactor system of Aspect 5, wherein the biologics coating comprises at least one of laminin, fibronectin, extracellular proteins, and synthetic peptides.

Aspect 7 pertains to the bioreactor system of any one of Aspects 1-6, wherein the polymer base comprises a thermopolymer.

Aspect 8 pertains to the bioreactor system of any one of Aspects 1-6, wherein the polymer base comprises at least one of polyethylene terephthalate, polypropylene, nylon, polylactic acid, polybenzimidazole, polycarbonate, polyether sulfone, polyoxymethylene, polyetherether ketone, polyethylene, polyphenylene oxide, polyphenylene sulfide, polyvinyl chloride, and polyvinylidene fluoride.

Aspect 9 pertains to the bioreactor system of any one of Aspects 1-8, wherein the substrate comprises woven or non-woven fibers.

Aspect 10 pertains to the bioreactor system of any one of Aspects 1-9, wherein the cell culture matrix comprises a plurality of layers of substrate.

Aspect 11 pertains to the bioreactor system of Aspect 10, wherein the plurality of layers is in a stacked arrangement.

Aspect 12 pertains to the bioreactor system of any one of Aspects 1-11, wherein the substrate comprises a rolled substrate.

Aspect 13 pertains to the bioreactor system of any one of Aspects 1-12, wherein the substrate comprises fibers comprising fiber diameter of from about 10 micrometers to about 500 micrometers.

Aspect 14 pertains to the bioreactor system of any one of Aspects 1-13, wherein the substrate comprises a regular array of openings between fibers of the substrate.

Aspect 15 pertains to the bioreactor system of any one of Aspects 1-14, wherein the substrate comprises an effective porosity of about 10% to about 80%.

Aspect 16 pertains to the bioreactor system of any one of Aspects 1-15, wherein the substrate is configured for at least one of viral vector production, induced pluripotent stem cells (iPSCs), mesenchymal stem cells (MSCs), protein production, antibody production, and extracellular vesicle production.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A bioreactor system for culturing cells, the system comprising:
a cell culture vessel comprising at least one interior reservoir configured for flowing liquid media therethrough; and
a cell culture matrix disposed in the reservoir, the cell culture matrix comprising a substrate comprises a polymer base and a polystyrene coating disposed on the polymer base.

2. The bioreactor system of claim 1, wherein the polystyrene coating is functionalized to enhance cell attachment and/or growth thereto.

3. The bioreactor system of claim 1, wherein the polystyrene coating is plasma treated.

4. The bioreactor system of claim 1, further comprising a cell attachment treatment on the polystyrene coating.

5. The bioreactor system of claim 4, wherein the cell attachment treatment comprises at least one of an oxygen plasma, an allylamine coating, a carboxylic acid group, or a biologics coating.

6. The bioreactor system of claim 5, wherein the biologics coating comprises at least one of laminin, fibronectin, extracellular proteins, and synthetic peptides.

7. The bioreactor system of claim 1, wherein the polymer base comprises a thermopolymer.

8. The bioreactor system of claim 1, wherein the polymer base comprises at least one of polyethylene terephthalate, polypropylene, nylon, polylactic acid, polybenzimidazole, polycarbonate, polyether sulfone, polyoxymethylene, polyetherether ketone, polyethylene, polyphenylene oxide, polyphenylene sulfide, polyvinyl chloride, and polyvinylidene fluoride.

9. The bioreactor system of claim 1, wherein the substrate comprises woven or non-woven fibers.

10. The bioreactor system of claim 1, wherein the cell culture matrix comprises a plurality of layers of substrate.

11. The bioreactor system of claim 10, wherein the plurality of layers is in a stacked arrangement.

12. The bioreactor system of claim 1, wherein the substrate comprises a rolled substrate.

13. The bioreactor system of claim 1, wherein the substrate comprises fibers comprising fiber diameter of from about 10 micrometers to about 500 micrometers.

14. The bioreactor system of claim 1, wherein the substrate comprises a regular array of openings between fibers of the substrate.

15. The bioreactor system of claim 1, wherein the substrate comprises an effective porosity of about 10% to about 80%.

16. The bioreactor system of claim 1, wherein the substrate is configured for at least one of viral vector production, induced pluripotent stem cells (iPSCs), mesenchymal stem cells (MSCs), protein production, antibody production, and extracellular vesicle production.
